(19) United States
(12) Patent Application Publication
(10) Pub. No.: US 2026/0102223 A1
(43) Pub. Date: Apr. 16, 2026

(54) DENTAL APPLIANCE, SYSTEM, AND METHOD

(71) Applicant: Solventum Intellectual Properties Company, Maplewood, MN (US)

(72) Inventors: Yuxin TONG, Lake Elmo, MN (US); Nicholas WREN, Woodbury, MN (US); Daniel J. SKAMSER, Ham Lake, MN (US); Zhihui MIAO, St. Paul, MN (US); Deborah SCHNEIDERMAN, St. Paul, MN (US); Ming-Lai LAI, Afton, MN (US)

(21) Appl. No.: 19/115,140

(22) PCT Filed: Oct. 3, 2023
(86) PCT No.: PCT/IB2023/059925

§ 371 Date: Mar. 25, 2025

Related U.S. Application Data
(60) Provisional application No. 63/416,126, filed on Oct. 14, 2022.

(51) Int. Cl. A61C 7/08

(52) U.S. Cl. CPC A61C 7/08; A61C 2204/00

(57) ABSTRACT

A dental appliance for a patient includes a body including a first surface and a second surface opposite to the first surface. The second surface defines a channel for receiving a plurality of teeth of the patient. The dental appliance further includes an arrangement of a plurality of features disposed on the first surface of the body. Each of the plurality of features includes a protrusion extending from the first surface opposite to the second surface; a depression extending partially from the first surface wards the second surface; or a through-hole extending from the first surface to the second surface. The arrangement of the plurality of features encodes a feature data representative of a dental appliance information of the dental appliance or a patient information of the patient.

### TECHNICAL FIELD

The present disclosure relates to a dental appliance for a patient, a system including at least one dental appliance for a corresponding patient and an imaging device, a method of forming the dental appliance, and a method of identifying the dental appliance.

### BACKGROUND

An orthodontic treatment is conducted by a dental practitioner for moving one or more teeth of a patient from a malposition to a desired position in a mouth of the patient. In some cases, an orthodontic appliance, for example, an orthodontic bracket and an aligner, may be used in the orthodontic treatment for moving the one or more teeth of the patient from the malposition to the desired position in a patient. The orthodontic appliance is therefore custom made for the patient according to an orthodontic treatment stage of the orthodontic treatment.

Nowadays, due to advancement in manufacturing techniques, a large number of orthodontic appliances for different patients can be mass-produced in a manufacturing facility. Further, each orthodontic appliance may have to be identified and tracked so that each of the orthodontic appliance is delivered to the correct patient.

### SUMMARY

In a first aspect, the present disclosure provides a dental appliance for a patient. The dental appliance includes a body including a first surface and a second surface opposite to the first surface. The second surface defines one or more cavities for receiving a plurality of teeth of the patient. The dental appliance further includes an arrangement of a plurality of features disposed on the first surface of the body. Each of the plurality of features includes a protrusion extending from the first surface opposite to the second surface; a depression extending partially from the first surface towards the second surface; or a through-hole extending from the first surface to the second surface. The arrangement of the plurality of features encodes feature data representative of a dental appliance information of the dental appliance or a patient information of the patient.

In a second aspect, the present disclosure provides a system. The system includes at least one dental appliance for a corresponding patient. The at least one dental appliance includes a body including a first surface and a second surface opposite to the first surface. The second surface defines a channel for receiving a plurality of teeth of the corresponding patient. The at least one dental appliance further includes an arrangement of a plurality of features disposed on the first surface of the body. Each of the plurality of features includes a protrusion extending from the first surface opposite to the second surface; a depression extending partially from the first surface towards the second surface; or a through-hole extending from the first surface to the second surface. The arrangement of the plurality of features encodes data representative of a dental appliance information of the dental appliance or a patient information of the patient. The system further includes an imaging device and a processor communicably coupled to the imaging device.

In a third aspect, the present disclosure provides a method of forming a dental appliance for a patient. The method includes obtaining an initial digital model of the dental appliance. The method further includes encoding a feature data into an arrangement of a plurality of features. Each of the plurality of features includes a protrusion, a depression, or a through-hole. The feature data is representative of a dental appliance information of the dental appliance or a patient information of the patient. The method further includes identifying one or more regions of the initial digital model for placement of the arrangement of the plurality of features. The method further includes incorporating the arrangement of the plurality of features in the one or more regions of the initial digital model to generate a digital model of the dental appliance. The method further includes forming the dental appliance based on the digital model of the dental appliance.

In a fourth aspect, the present disclosure provides a method of identifying a dental appliance for a patient. The method further includes receiving the one or more images of the at least one dental appliance. The method further includes identifying an arrangement of a plurality of features of the at least one dental appliance based on the one or more images. Each of the plurality of features includes a protrusion, a depression, or a through-hole. The method further includes decoding the arrangement of the plurality of features using decoding data to determine feature data encoded by the arrangement of the plurality of features. The feature data is representative of a dental appliance information of the dental appliance or a patient information of the patient.

The details of one or more examples of the disclosure are set forth in the accompanying drawings and the description below. Other features, objects, and advantages of the disclosure will be apparent from the description and drawings, and from the claims.

### BRIEF DESCRIPTION OF THE DRAWINGS

Exemplary embodiments disclosed herein may be more completely understood in consideration of the following detailed description in connection with the following figures. The figures are not necessarily drawn to scale. Like numbers used in the figures refer to like components. However, it will be understood that the use of a number to refer to a component in a given figure is not intended to limit the component in another figure labeled with the same number.

FIG. 1 a schematic top view of an exemplary dental arch of a patient undergoing an orthodontic treatment;

FIG. 2A shows a schematic bottom perspective view of a dental appliance for the patient, according to an embodiment of the present disclosure;

FIG. 2B shows a schematic top view of the dental appliance, according to an embodiment of the present disclosure;

FIG. 2C shows a magnified schematic top view of a portion of the dental appliance, according to an embodiment of the present disclosure;

FIG. 3 shows a schematic top view of an arrangement of a plurality of features disposed on the dental appliance, according to an embodiment of the present disclosure;

FIG. 4A shows a schematic top view of the dental appliance, according to an embodiment of the present disclosure;

FIG. 4B shows a magnified schematic top view of a portion of the dental appliance of FIG. 4A, according to an embodiment of the present disclosure;

FIG. 4C shows a schematic top view of the dental appliance, according to another embodiment of the present disclosure;

FIG. 4D shows a magnified schematic top view of a portion of the dental appliance of FIG. 4C, according to an embodiment of the present disclosure;

FIG. 5 shows a magnified portion of a photograph of the dental appliance, according to an embodiment of the present disclosure;

FIG. 6 shows a schematic block diagram of a system, according to an embodiment of the present disclosure;

FIG. 7 shows a schematic top view of the system, according to an embodiment of the present disclosure;

FIG. 8 shows a flowchart illustrating a method of forming the dental appliance, according to an embodiment of the present disclosure;

FIGS. 9A-9E show schematic views of various steps of forming the dental appliance, according to an embodiment of the present disclosure; and

FIG. 10 shows a flowchart illustrating a method of identifying the dental appliance for the patient, according to an embodiment of the present disclosure.

### DETAILED DESCRIPTION

In the following description, reference is made to the accompanying figures that form a part thereof and in which various embodiments are shown by way of illustration. It is to be understood that other embodiments are contemplated and may be made without departing from the scope or spirit of the present disclosure. The following detailed description, therefore, is not to be taken in a limiting sense.

In the following disclosure, the following definitions are adopted.

As used herein, all numbers should be considered modified by the term “about”. As used herein, “a,” “an,” “the,” “at least one,” and “one or more” are used interchangeably.

As used herein as a modifier to a property or attribute, the term “generally”, unless otherwise specifically defined, means that the property or attribute would be readily recognizable by a person of ordinary skill but without requiring absolute precision or a perfect match (e.g., within +/−20 % for quantifiable properties).

The term “substantially”, unless otherwise specifically defined, means to a high degree of approximation (e.g., within +/−10% for quantifiable properties) but again without requiring absolute precision or a perfect match.

The term “about”, unless otherwise specifically defined, means to a high degree of approximation (e.g., within +/−5% for quantifiable properties) but again without requiring absolute precision or a perfect match.

Terms such as same, equal, uniform, constant, strictly, and the like, are understood to be within the usual tolerances or measuring error applicable to the particular circumstance rather than requiring absolute precision or a perfect match.

As used herein, the terms “first” and “second” are used as identifiers. Therefore, such terms should not be construed as limiting of this disclosure. The terms “first” and “second” when used in conjunction with a feature or an element can be interchanged throughout the embodiments of this disclosure.

As used herein, “at least one of A and B” should be understood to mean “only A, only B, or both A and B”.

As used herein, when a first material is termed as “similar” to a second material, at least 90 weight % of the first and second materials are identical and any variation between the first and second materials comprises less than about 10 weight % of each of the first and second materials.

As used herein, the term “processor” refers to a computing device that couples to one or more other devices/circuits, e.g., switching circuits, etc., and which may be configured to communicate with, e.g., to control, such devices/circuits. The processor may include any device that performs logic operations. A processor may include a general processor, a central processing unit, an application specific integrated circuit (ASIC), a digital signal processor, a field programmable gate array (FPGA), a digital circuit, an analog circuit, a microcontroller, any other type of controller, or any combination thereof.

As used herein, the term “communicably coupled” refers to direct coupling between components and/or indirect coupling between components via one or more intervening components. Such components and intervening components may include, but are not limited to, junctions, communication paths, components, circuit elements, circuits, functional blocks, and/or devices. As an example of indirect coupling, a signal conveyed from a first component to a second component may be modified by one or more intervening components by modifying the form, nature, or format of information in a signal, while one or more elements of the information in the signal are nevertheless conveyed in a manner than can be recognized by the second component.

As used herein, the term “three-dimensional representation,” refers to any three-dimensional surface map of an object, such as a point cloud of surface data, a set of two-dimensional polygons, or any other data representing all or some of the surface of an object, as might be obtained through the capture and/or processing of three-dimensional scan data, unless a different meaning is explicitly provided or otherwise clear from the context. A “three-dimensional representation” may include volumetric and other representations, unless a different meaning is explicitly provided or otherwise clear from the context.

An orthodontic treatment is conducted by a dental practitioner for moving one or more teeth of a patient from a malposition to a desired position in a mouth of the patient. The orthodontic treatment may improve a facial appearance of the patient. In some cases, the orthodontic treatment may also improve function of the one or more teeth by providing improved occlusion during mastication. In some cases, an orthodontic appliance, for example, a clear tray aligner (CTA), may be used in the orthodontic treatment for moving the one or more teeth of the patient from the malposition to the desired position in a patient. The orthodontic appliance is therefore custom made for the patients according to an orthodontic treatment stage of the orthodontic treatment.

While CTAs may be created in a practitioner's office or at a dental lab, the majority of CTAS are mass-produced in remote manufacturing facilities. Given the case volume and the custom nature of the treatment, each orthodontic appliance must be identified and tracked so that each of the orthodontic appliance is ultimately delivered to the correct practitioner or patient.

Some conventional techniques to identify and track orthodontic appliances use a unique QR code impressed on an excess area of each orthodontic appliance or a unique laser marking on each orthodontic appliance. However, impressing the unique QR code or generating the unique laser marking on the orthodontic appliance may add cost and/or complexity to the manufacturing techniques. This may also increase a manufacturing time of each of the orthodontic appliance.

Therefore, a suitable solution may be required which may reduce the additional cost and/or complexity associated with the conventional techniques to identify and track each orthodontic appliance.

The present disclosure relates to a dental appliance for a patient, a system including at least one dental appliance for a corresponding patient and an imaging device, a method of forming the dental appliance, and a method of identifying the dental appliance.

The dental appliance includes a body including a first surface and a second surface opposite to the first surface. The second surface defines a channel for receiving a plurality of teeth of the patient. The dental appliance further includes an arrangement of a plurality of features disposed on the first surface of the body. Each of the plurality of features includes a protrusion extending from the first surface opposite to the second surface; a depression extending partially from the first surface towards the second surface; or a through-hole extending from the first surface to the second surface. The arrangement of the plurality of features encodes feature data representative of a dental appliance information of the dental appliance or a patient information of the patient.

The arrangement of the plurality of features that encodes the feature data may be readily decoded to determine the feature data encoded by the arrangement of the plurality of features. In another embodiment, the plurality of features may encode a unique identifier suitable for database lookup of appliance and patient information. The features are spaced apart from each other to encode the feature data. Thus, the arrangement of the plurality of features may be used to identify and track each dental appliance without a need to add any support structure. This may reduce a material requirement for manufacturing the dental appliance and may further decrease a manufacturing cost of the dental appliance. This may further reduce wastage as typically the support structures are required to be removed and discarded. Therefore, manufacturing the dental appliance may also be environmentally sustainable. Moreover, a manufacturing complexity of the dental appliance may be reduced as the arrangement of the plurality of features are incorporated in a design of the dental appliance, and therefore do not require a separate marking or labeling step.

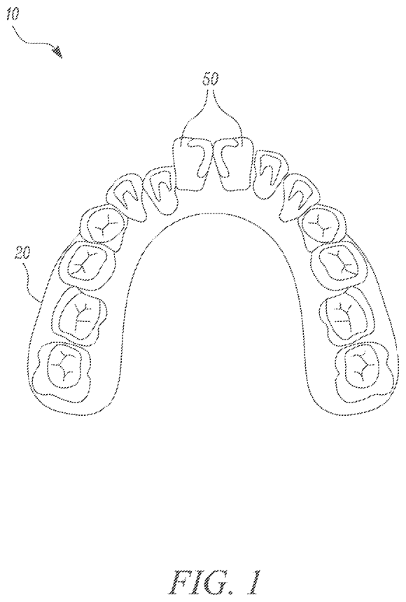

Referring now to figures, FIG. 1 illustrates a schematic top view of an exemplary dental arch 20 of a patient 10 undergoing an orthodontic treatment. It may be noted that the dental arch 20 shown in FIG. 1 is a lower dental arch of the patient 10. In some other embodiments, the dental arch 20 may include an upper dental arch of the patient 10. The dental arch 20 includes a plurality of teeth 50. In some cases, at least one tooth 50 of the plurality of teeth 50 of the patient 10 may be malpositioned. Therefore, the patient 10 may be required to undergo the orthodontic treatment to correct malpositioning of the at least one tooth 50. The at least one tooth 50 may be of the lower dental arch or the upper dental arch of the patient 10.

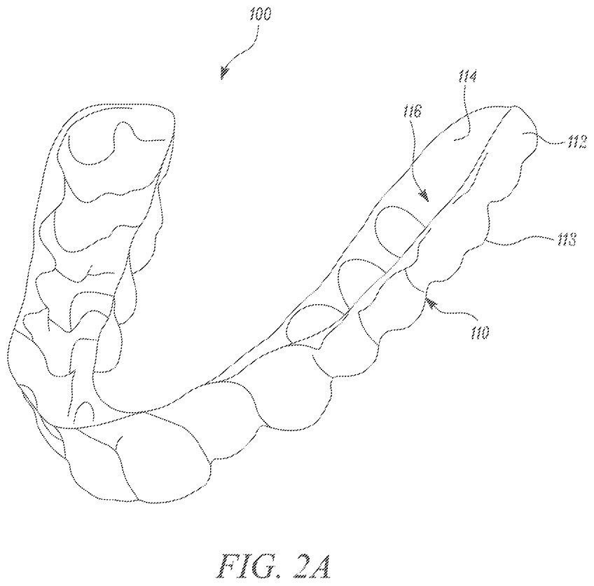

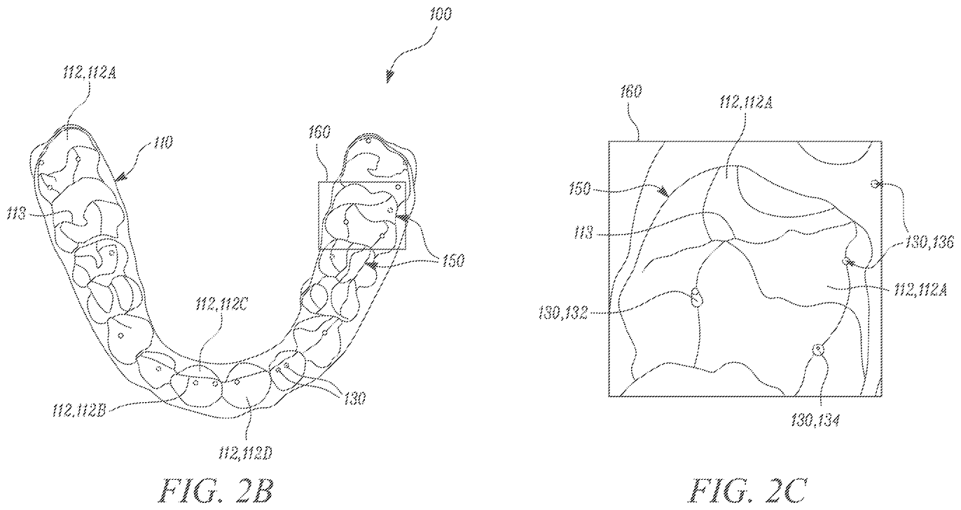

FIG. 2A illustrates a schematic bottom perspective view of a dental appliance 100 for the patient 10 shown in FIG. 1, according to an embodiment of the present disclosure. FIG. 2B illustrates a schematic top view of the dental appliance 100, according to an embodiment of the present disclosure. FIG. 2C is a magnified schematic top view of a portion 160 of the dental appliance 100 of FIG. 2B, according to an embodiment of the present disclosure.

Referring to FIGS. 2A to 2C, in some embodiments, the dental appliance 100 is at least one of an aligner, retainer, or bonding tray. In the illustrated embodiment of FIGS. 2A-2C, the dental appliance 100 is an aligner. In some other embodiments, the dental appliance 100 may be a retainer. The dental appliance 100 may be any appliance configured to apply forces and/or moments on the at least one tooth 50 (shown in FIG. 1) to move the at least one tooth 50 from an initial arrangement (i.e., a malposition) to a target arrangement (i.e., a desired position) in a dentition of the patient 10 (shown in FIG. 1) undergoing the orthodontic treatment. The dental appliance 100 may apply the forces and/or the moments on the at least one tooth 50 according to an orthodontic treatment stage of the patient 10. Therefore, the dental appliance 100 is generally custom made for the patient 10 and is unique for the patient 10.

The dental appliance 100 includes a body 110. The body 110 includes a first surface 112 and a second surface 114 opposite to the first surface 112. The second surface 114 includes one or more cavities defining channel 116 for receiving the plurality of teeth 50 (shown in FIG. 1) of the patient 10. The channel 116 receives and may resiliently reposition the plurality of teeth 50 in accordance with the orthodontic treatment stage.

The first surface 112 has a contour 113. The contour 113 represents the plurality of teeth 50 of the patient 10. Specifically, the contour 113 may represent an outer contour of the plurality of teeth 50 of the patient 10. In other words, the contour 113 may substantially conform to the outer surface of the plurality of teeth 50 of the patient 10.

In some embodiments, the body 110 of the dental appliance 100 may include biocompatible materials, such as a polyester, a co-polyester, a polycarbonate, a polycarbonate, a thermoplastic polyurethane, a polypropylene, a polyethylene, a polypropylene and polyethylene copolymer, an acrylic, a cyclic block copolymer, a polyetheretherketone, a polyamide, a polyethylene terephthalate, a polybutylene terephthalate, a polyetherimide, a polyethersulfone, a polytrimethylene terephthalate, a styrenic block copolymer (SBC), a silicone rubber, an elastomeric alloy, a thermoplastic elastomer (TPE), a thermoplastic vulcanizate (TPV) elastomer, a polyurethane elastomer, a block copolymer elastomer, a polyolefin blend elastomer, a thermoplastic co-polyester elastomer, a thermoplastic polyamide elastomer, or combinations thereof. In some embodiments, the materials used for fabrication of the body 110 may be provided in an uncured form (e.g., as a liquid, resin, powder, etc.) and can be cured (e.g., by photopolymerization, light curing, gas curing, laser curing, crosslinking, etc.). The properties of the material before curing may differ from the properties of the material after curing. In some embodiments, the material may be a substantially transparent material.

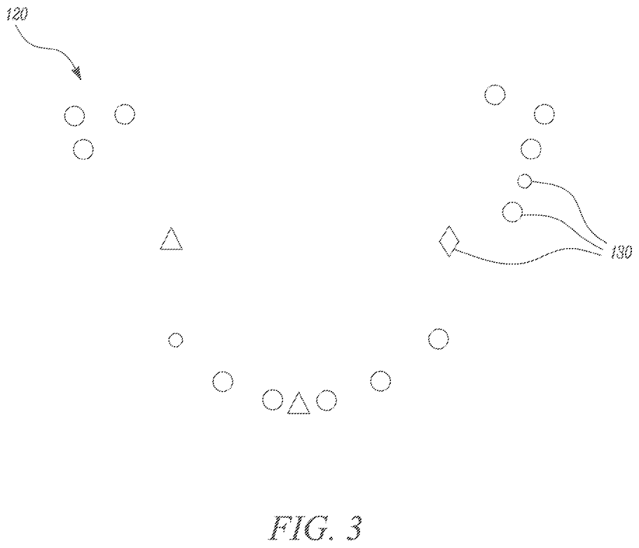

The dental appliance 100 further includes an arrangement 120 (shown separately in FIG. 3) of a plurality of features 130 disposed on the first surface 112 of the body 110. The features 130 are spaced apart from each other. In some embodiments, the first surface 112 includes an occlusal surface 112A of the dental appliance 100 or an incisal surface 112B of the dental appliance 100. In some embodiments, the first surface 112 may include a lingual surface 112C of the dental appliance 100 or a facial surface 112D of the dental appliance 100. The facial surface 112D of the dental appliance 100 may include a labial surface or a buccal surface.

Each of the plurality of features 130 includes a protrusion 132 extending from the first surface 112 opposite to the second surface 114 (shown in FIG. 2A), a depression 134 extending partially from the first surface 112 towards the second surface 114, or a through-hole 136 extending from the first surface 112 to the second surface 114.

In some embodiments, each of the plurality of features 130 including the through-hole 136 and disposed at the occlusal surface 112A of the dental appliance 100 or the incisal surface 112B of the dental appliance 100 may provide improved performance, especially in comfort and hygiene.

In some embodiments, the first surface 112 includes a plurality of regions (e.g., quadrants) 150 corresponding to and aligned with the plurality of teeth 50 of the patient 10 shown in FIG. 1. In some embodiments, the features 130 are arranged in at least two regions 150 of the plurality of regions 150.

FIG. 3 is a schematic top view of the arrangement 120 of the plurality of features 130 disposed on the first surface 112 (shown in FIGS. 2A-2C), according to an embodiment of the present disclosure.

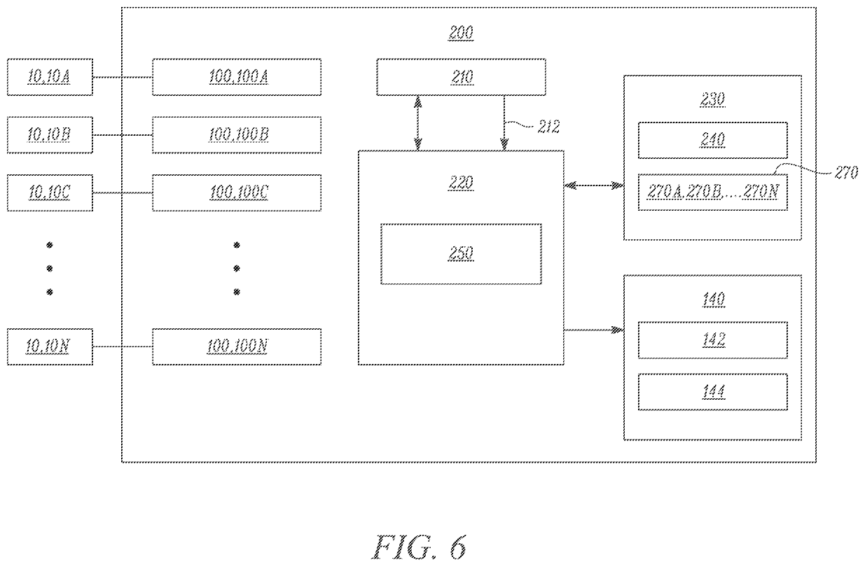

Referring to FIGS. 1 to 3, the arrangement 120 of the plurality of features 130 encodes feature data 140 (shown in FIG. 6) representative of a dental appliance information 142 (shown in FIG. 6) of the dental appliance 100 or a patient information 144 (shown in FIG. 6) of the patient 10.

In some embodiments, the dental appliance information 142 includes at least one of a production batch information of the dental appliance 100, a material information of the dental appliance 100, and a manufacturing timestamp information of the dental appliance 100. For example, the dental appliance information 142 may include a part number of the dental appliance 100, a date of manufacture of the dental appliance 100, a production time of the dental appliance 100, a resin batch identifier of the dental appliance 100, a clinician (e.g., practitioner or technician) name/identifier of the dental appliance 100, a process equipment identifier of the dental appliance 100, and so forth.

Thus, the arrangement 120 of the plurality of features 130 encoding the feature data 140 may allow tracking of each dental appliance 100 throughout a manufacturing process and allow tying each dental appliance 100 to equipment and operators associated with its manufacturing process. Therefore, any manufacturing defects may be traceable to materials, processes, and/or equipment.

In some embodiments, the patient information 144 includes at least one of an identification information of the patient 10, an address information of the patient 10, a contact information of the patient 10, and a treatment stage information of the patient 10. For example, the patient information 144 may include a patient name of the patient 10, a patient identifier of the patient 10, a case number of the patient 10, and so forth. Thus, the arrangement 120 of the plurality of features 130 encoding the feature data 140 may allow each dental appliance 100 to be uniquely mapped to a particular patient (e.g., the patient 10).

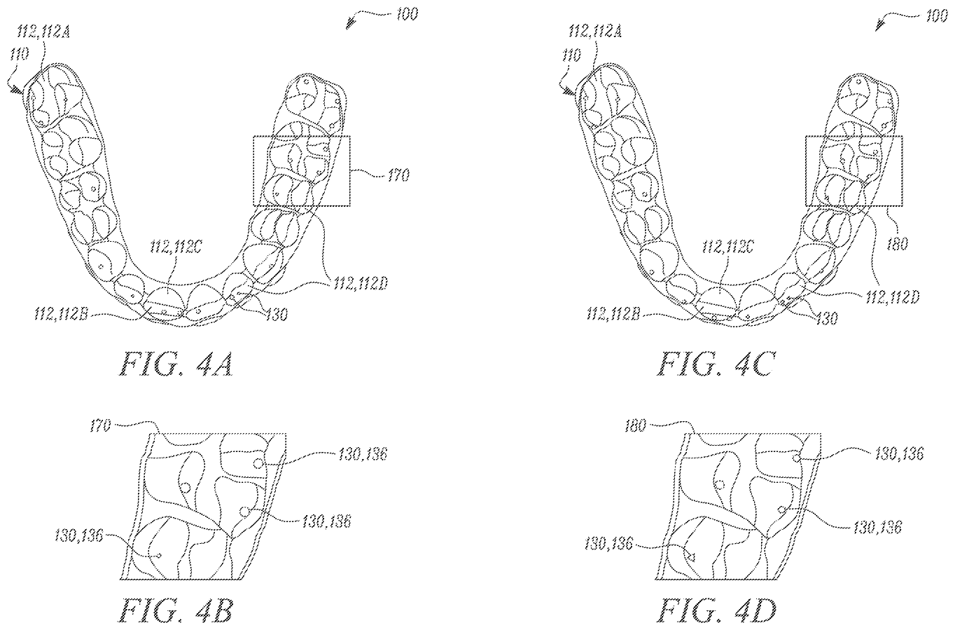

FIG. 4A illustrates a schematic top view of the dental appliance 100, according to an embodiment of the present disclosure. FIG. 4B is a magnified schematic top view of a portion 170 of the dental appliance 100 of FIG. 4A, according to an embodiment of the present disclosure. FIG. 4C illustrates a top view of the dental appliance 100, according to another embodiment of the present disclosure. FIG. 4D is a magnified schematic top view of a portion 180 of the dental appliance 100 of FIG. 4C, according to an embodiment of the present disclosure.

Referring to FIGS. 4A to 4D, in some embodiments, at least one feature 130 of the plurality of features 130 has a different shape or different dimensions than other features 130 of the plurality of features 130. For example, as is apparent from the portion 170 of FIG. 4B, some features 130 of the plurality of features 130 have different dimensions from other features 130 of the plurality of features 130. In a further example, as is apparent from the portion 180 of FIG. 4D, some features 130 of the plurality of features 130 have different shapes from other features 130 of the plurality of features 130. In some embodiments, at least one feature 130 of the plurality of features 130 is the through-hole 136 having a circular shape, an elliptical shape, a triangular shape, a rectangular shape, or a polygonal shape.

Thus, different design parameters (e.g., a shape, a size, and a position) of the features 130 may therefore generate different and unique arrangements 120 to identify or track the dental appliance 100 without any requirement of additional labels (e.g., QR codes and laser markings). The different arrangements 120 may then be recognized by a system 200 (shown in FIG. 6).

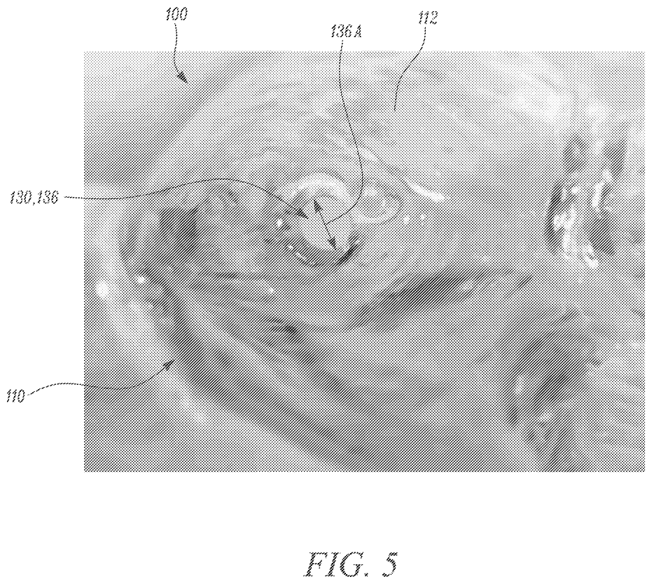

FIG. 5 illustrates a magnified portion of a photograph of the dental appliance 100, according to an embodiment of the present disclosure.

In the illustrated embodiment of FIG. 5, at least one feature 130 of the plurality of features 130 is the through-hole 136 having a circular shape. In such embodiments, a maximum width 136A of the through-hole 136 is a diameter of the through-hole 136. Further, in some embodiments, the maximum width 136A of the through-hole 136 is less than or equal to 4 millimeters (mm). In some embodiments, the maximum width 136A of the through-hole 136 is less than or equal to about 3.5 mm, about 3.0 mm, about 2.5 mm, about 2.0 mm, about 1.5 mm, or about 1.0 mm. In some embodiments, the maximum width 136A of the through-hole 136 is less than or equal to about 0.5 mm, about 0.25 mm, or about 0.1 mm. Therefore, the through-hole 136 may be invisible to the naked eye and yet be recognized by the system 200 (shown in FIG. 6). Therefore, this may further reduce a considerable amount of cost compared to conventional labeling methods.

FIG. 6 illustrates a schematic block diagram of the system 200, according to an embodiment of the present disclosure. In some embodiments, the system 200 may be a machine vision-based identification system.

The system 200 includes at least one dental appliance 100 for a corresponding patient 10. In the illustrated embodiment of FIG. 6, the at least one dental appliance 100 includes a plurality of dental appliances 100A-100N corresponding to a plurality of corresponding patients 10A-10N. For example, the dental appliance 100A corresponds to the corresponding patient 10A, the dental appliance 100B corresponds to the corresponding patient 10B, the dental appliance 100C corresponds to the corresponding patient 10C, and so on. The at least one dental appliance 100 may include any number of dental appliances corresponding to any number of corresponding patients, as per desired application attributes. In other embodiments, the dental appliance 100A corresponds to a first phase of treatment for a patient, appliance 100B corresponds to a second phase of treatment for the same patient, appliance 100C corresponds to a third phase of treatment for that same patient, and so on. The appliances 100 of the system 200 can be a combination of multiple appliances for a single patient and appliances for disparate patients.

Referring to FIGS. 1-6, the at least one dental appliance 100 includes the body 110 including the first surface 112 and the second surface 114 opposite to the first surface 112. The second surface 114 defines the channel 116 for receiving the plurality of teeth 50 of the corresponding patient 10. The at least one dental appliance 100 further includes the arrangement 120 of the plurality of features 130 disposed on the first surface 112 of the body 110.

As discussed above, each of the plurality of features 130 includes the protrusion 132 extending from the first surface 112 opposite to the second surface 114, the depression 134 extending partially from the first surface 112 towards the second surface 114, or the through-hole 136 extending from the first surface 112 to the second surface 114. In some embodiments, the first surface 112 includes the one or more regions 150 corresponding to and aligned with the plurality of teeth 50 of the corresponding patient 10. In some embodiments, the plurality of features 130 is arranged in at least two regions 150 of the plurality of regions 150.

Further, the arrangement 120 of the plurality of features 130 encodes the feature data 140 representative of the dental appliance information 142 of the at least one dental appliance 100 or the patient information 144 of the corresponding patient 10. In some embodiments, the dental appliance information 142 includes at least one of the production batch information of the at least one dental appliance 100, the material information of the at least one dental appliance 100, and the manufacturing timestamp information of the at least one dental appliance 100. In some embodiments, the patient information 144 includes at least one of the identification information of the corresponding patient 10, the address information of the corresponding patient 10, the contact information of the corresponding patient 10, and the treatment stage information of the corresponding patient 10.

The system 200 further includes an imaging device 210. The imaging device 210 may include any suitable lens and sensor assembly, as per desired application attributes. In some embodiments, the imaging device 210 includes at least one of a red-green-blue (RGB) sensor, a black and white sensor, a near infrared (NIR) sensor, an imaging array, and a depth sensor. The depth sensor may further enable the imaging device 210 to capture three-dimensional data (e.g., depth data) in addition to two-dimensional data.

The system 200 further includes a processor 220 communicably coupled to the imaging device 210. In some embodiments, the processor 220 may include any suitable data processor for processing data. For example, the processor 220 may include a microprocessor, a microcontroller, a computer, or other suitable devices that control operation of devices and execute programs. Various other examples of the processor 220 include central processing units (“CPUs”), microcontrollers, programmable logic devices, field programmable gate arrays, digital signal processing (“DSP”) devices, and the like. The processor 220 may include any general variety device such as a reduced instruction set computing (“RISC”) device, a complex instruction set computing (“CISC”) device, or a specially designed processing device, such as an application-specific integrated circuit (“ASIC”) device. The processor 220 may execute computer executable instructions or computer code embodied in a memory 230. In some embodiments, the computer executable instructions or computer code are stored in the memory 230. When the computer executable instructions or computer code are executed by the processor 220, the computer executable instructions or computer code cause the processor 220 to perform one or more of the actions, operations, methods, or functions described herein.

In some embodiments, the system 200 further includes the memory 230. The memory 230 is communicably coupled to the processor 220. The memory 230 may include any volatile or non-volatile storage element, for example, random access memory (RAM), such as synchronous dynamic random access memory (SDRAM), read-only memory (ROM), non-volatile random access memory (NVRAM), electrically erasable programmable read-only memory (EEPROM), and FLASH memory. In some embodiments, the memory 230 may include an external storage device, such as a hard-disk, magnetic tape, a magnetic or optical data storage media, such as a compact disk (CD), a digital versatile disk (DVD), a Blu-ray disk, a holographic data storage media, and the like.

In some embodiments, the system 200 may include a computing device (not shown) including the processor 220 and the memory 230. In other words, in some embodiments, the computing device includes the processor 220 communicably coupled to the imaging device 210 and the memory 230 communicably coupled to the processor 220. The computing device may include, for example, a desktop computer, a laptop, a smartphone, and the like.

The memory 230 stores a decoding data 240 for decoding the arrangement 120 of the plurality of features 130 of the at least one dental appliance 100. The decoding data may function as an algorithm to identify the feature data, or may cause a database lookup to match the encoded feature data to appliance or patient information. For instance, the feature data may comprise a generic identification number that corresponds an identifier stored in a processor-accessible database. The decode process could entail match the generic ID encoded in the feature data with an identifier in the database, which would itself correspond to patient and/or appliance information. In some embodiments, the memory 230 further stores at least one digital model 270A-270N (collectively and individually, the digital model 270) corresponding to the at least one dental appliance 100. For example, the memory 230 may store the digital model 270A corresponding to the dental appliance 100A, the digital model 270B corresponding to the dental appliance 100B, the digital model 270C corresponding to the dental appliance 100C, and so on.

In some embodiments, the processor 220 is configured to control the imaging device 210 to capture one or more images 212 of the at least one dental appliance 100. The processor 220 may control the imaging device 210 via a wired connection and/or a wireless connection.

In some embodiments, the processor 220 is further configured to receive the one or more images 212 of the at least one dental appliance 100 from the imaging device 210. The processor 220 may receive the one or more images 212 from the imaging device 210 via a wired connection and/or a wireless connection.

In some embodiments, the processor 220 is further configured to identify the arrangement 120 of the plurality of features 130 of the at least one dental appliance 100 based on the one or more images 212.

In some embodiments, the processor 220 is configured to execute a machine learning model 250 that is trained to receive the one or more images 212 of the at least one dental appliance 100 from the imaging device 210 and identify the arrangement 120 of the plurality of features 130 of the at least one dental appliance 100 based on the one or more images 212. In some embodiments, the machine learning model 250 is further trained to decode the arrangement 120 of the plurality of features 130 using the decoding data or algorithm 240 to determine the feature data 140. In some embodiments, the machine learning model 250 may include a convolutional neural network model. In some embodiments, the processor 220 may be configured to execute a predictive model to identify the arrangement 120 of the plurality of features 130 of the at least one dental appliance 100 based on the one or more images 212.

In some embodiments, the processor 220 is further configured to decode the arrangement 120 of the plurality of features 130 using the decoding data 240 to determine the feature data 140 encoded by the arrangement 120 of the plurality of features 130. In some cases, the decoded feature data 140 may be uploaded to an online portal. This may enable the corresponding patient 10 to track their dental appliance 100 in real-time. In some cases, the corresponding patient 10 may even upload an image (e.g., the one or more images 212) to the online portal to verify the feature data 140 (i.e., the dental appliance information 142 and the patient information 144).

In some embodiments, the processor 220 is configured to decode the arrangement 120 of the plurality of features 130 using the decoding data 240 to determine the treatment stage information of the corresponding patient 10. In some embodiments, the processor 220 is configured to determine the contour 113 of at least one portion of the first surface 112 of the at least one dental appliance 100 based on the one or more images 212. In some embodiments, the processor 220 is configured to compare the contour 113 of the at least one portion of the first surface 112 with the at least one digital model 270 and determine the identification information (e.g., the patient identifier, the patient name, etc.) of the corresponding patient 10 based on the comparison.

In some embodiments, upon decoding the arrangement 120 of the plurality of features 130 of each of the plurality of dental appliances 100, the processor 220 is further configured to group the plurality of dental appliances 100 according to the feature data 140. For example, the plurality of dental appliances 100 may be grouped according to the production batch information, the material information, and so forth. This may facilitate quality control. In some cases, the plurality of dental appliances 100 may be grouped according to the address information. This may facilitate packaging and delivery of the dental appliances 100. In some cases, the plurality of dental appliances 100 may be grouped for post-processing or sorting processes.

Further, grouping the plurality of dental appliances 100 according to the feature data 140 may reduce the amount of time to perform the quality control, thereby decreasing lead-time of the dental appliances 100. Decreasing the lead-time of the dental appliances 100 may further ensure delivery of the dental appliances 100 to the corresponding patients 10, as scheduled.

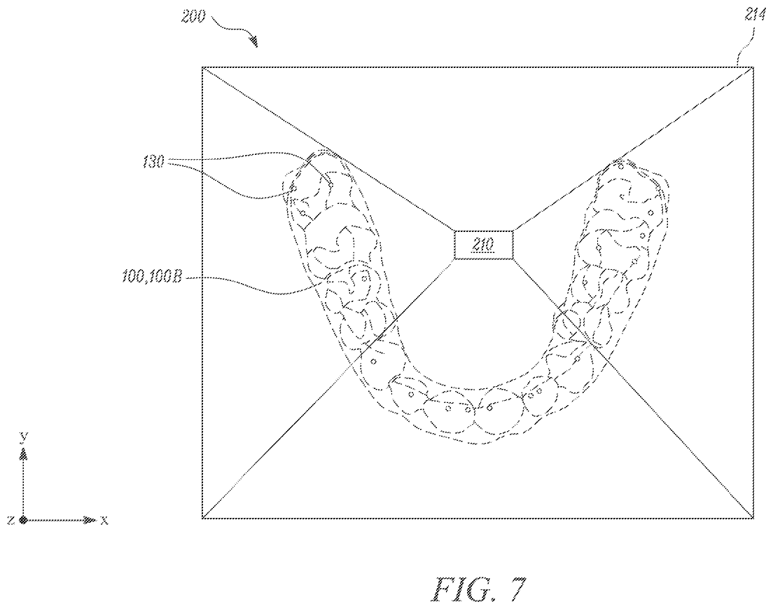

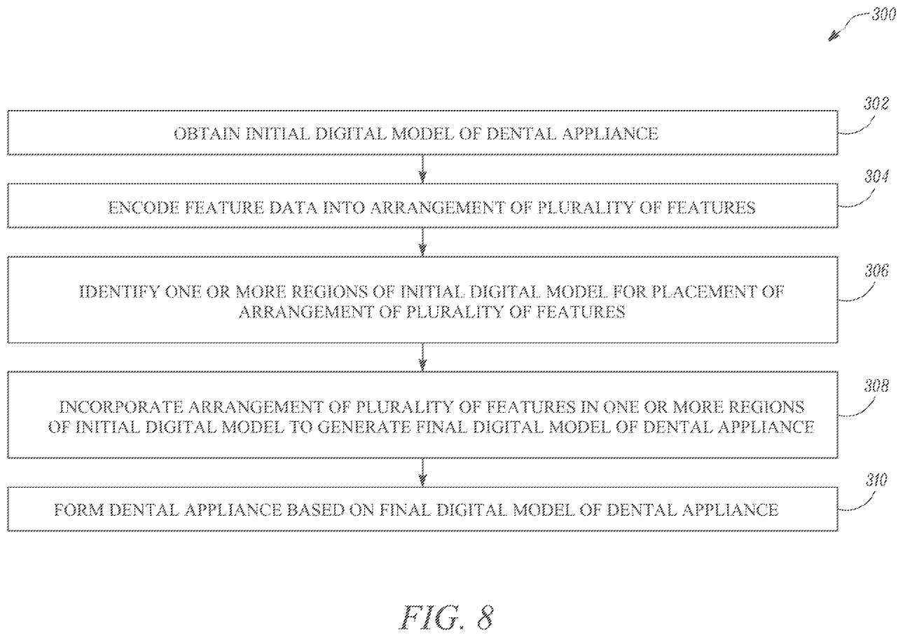

FIG. 7 illustrates a schematic top view of the system 200, according to an embodiment of the present disclosure. In some embodiments, the imaging device 210 is configured to capture the one or more images 212 (shown in FIG. 6) in a single projection plane 214. Specifically, in cases each of the plurality of features 130 is disposed at the occlusal surface 112A (shown in FIG. 2B) of the dental appliance 100 or the incisal surface 112B (shown in FIG. 2b) of the dental appliance 100, the imaging device 210 may capture the one or more images 212 in the single projection plane 214 and may not require capturing the one or more images 212 from several projections. Therefore, the imaging device 210 may have a fixed setup. This may eliminate a need for adjustable positioning to obtain the one or more images 212 from different angles or projections.

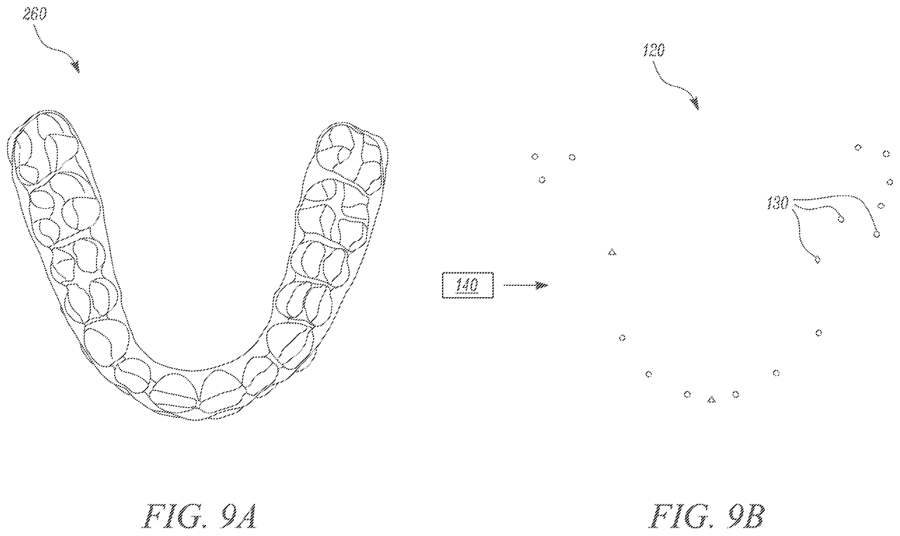

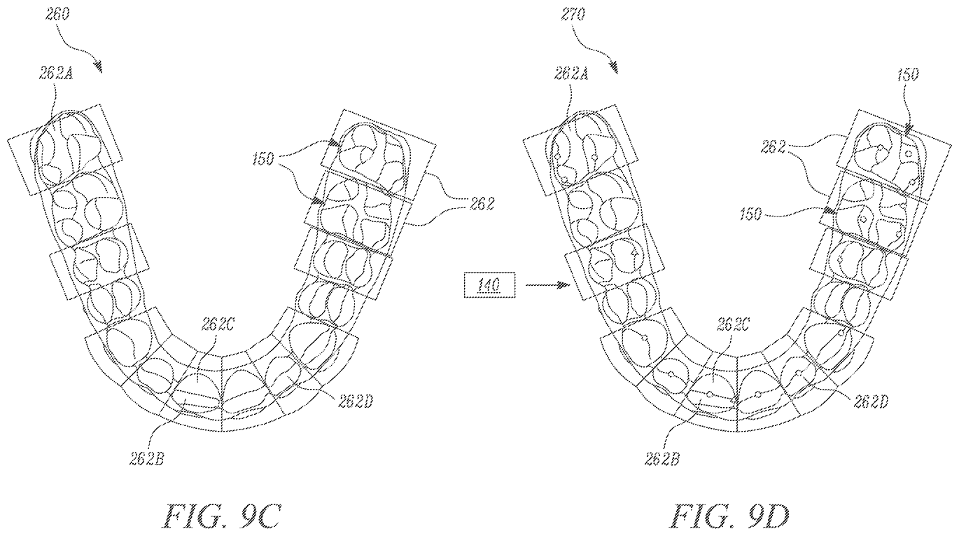

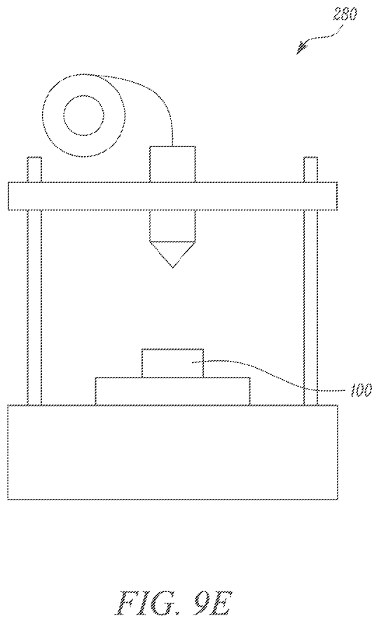

FIG. 8 illustrates a flowchart illustrating a method 300 of forming the dental appliance 100 for the patient 10, according to an embodiment of the present disclosure. FIGS. 9A-9E show schematic views of various steps of forming the dental appliance 100, according to an embodiment of the present disclosure. The method 300 will be described with reference to FIGS. 1 to 9A-9E. The method 300 includes the following steps:

FIG. 9A shows a schematic top view of an initial digital model 260 of the dental appliance 100 shown in FIG. 2B. At step 302, the method 300 includes obtaining the initial digital model 260 of the dental appliance 100. In some embodiments, the method 300 includes obtaining the initial digital model 260 of the dental appliance 100 by the processor 220 (shown in FIG. 6). In some embodiments, the initial digital model 260 may be a three-dimensional representation of the dental appliance 100. In some embodiments, the initial digital model 260 of the dental appliance 100 may be represented in a computer aided drafting (CAD) file or a 3D printable file, such as a stereolithography (STL) file.

In some embodiments, the initial digital model 260 may be based on a scanned data representative of the outer contour of the plurality of teeth 50 (shown in FIG. 1). In some embodiments, dental practitioners may optically scan the plurality of teeth 50 or a dental arch of the patient 10 undergoing the orthodontic treatment to generate the scanned data representative of the outer contour of the plurality of teeth 50. Therefore, in some embodiments, obtaining the initial digital model 260 of the dental appliance 100 may include optically scanning the plurality of teeth 50. In some embodiments, optically scanning the plurality of teeth 50 may include performing an intraoral scan. In some embodiments, optically scanning the plurality of teeth 50 may include performing a digital data capture, a computed tomography (CT), or a computer-aided tomography (CAT) of a mouth of the patient 10. In some other embodiments, optically scanning the plurality of teeth 50 may include indirectly performing a digital data capture of the mouth of the patient 10 by performing the digital data capture of a plaster model of the mouth of the patient 10 or of a dental impression of the mouth of the patient 10, rather than directly capturing a three-dimensional structure of the mouth of the patient 10. In the case of using the dental impression, the digital data capture may be inverted from a negative volume to a positive volume. In some embodiments, the scanned data representative of the outer contour of the plurality of teeth 50 is processed to generate the initial digital model 260 of the dental appliance 100.

In some embodiments, the initial digital model 260 may be based on a digital data from a database. Therefore, in some embodiments, obtaining the initial digital model 260 of the dental appliance 100 may include retrieving the digital data from the database. In some embodiments, the digital data may be adapted to generate the initial digital model 260 of the dental appliance 100. In some embodiments, the digital data may be provided by a file history of the patient 10 or from a previous digital data capture of the mouth of the patient 10. Exemplary methods of direct printing clear tray aligners and other resilient orthodontic apparatuses are set forth in U.S. Pat. No. 11,225,535 (Klun et al.) PCT Publication Nos. WO2016/109660 (Raby et al.), WO2016/148960 (Cinader et al.), and WO2016/149007 (Oda et al.) as well as US Publication Nos. US2011/0091832 (Kim, et al.), US2013/0095446 (Kitching).

At step 304, the method 300 includes encoding the feature data 140 into the arrangement 120 of the plurality of features 130 as shown in FIG. 9B. As discussed above, each of the plurality of features 130 may include the protrusion 132, the depression 134, or the through-hole 136. The feature data 140 is representative of the dental appliance information 142 of the dental appliance 100 or the patient information 144 of the patient 10.

FIG. 9C shows one or more regions 262 of the initial digital model 260 suitable for placement of the arrangement 120 of the plurality of features 130. At step 306, the method 300 includes identifying the one or more regions 262 of the initial digital model 260 for placement of the arrangement 120 of the plurality of features 130. In some embodiments, identifying the one or more regions 262 further includes identifying the one or more regions 262 on an occlusal surface 262A of the initial digital model 260 or an incisal surface 262B of the initial digital model 260. In some embodiments, identifying the one or more regions 262 further includes identifying the one or more regions 262 on a lingual surface 262C of the initial digital model 260 or a facial surface 262D of the initial digital model 260.

In some embodiments, the initial digital model 260 includes the plurality of teeth regions 150 corresponding to and aligned with the plurality of teeth 50 of the patient 10. In some embodiments, identifying the one or more regions 262 further includes identifying at least two teeth regions 150 of the plurality of teeth regions 150 for placement of the plurality of features 130 (shown in FIG. 9B).

FIG. 9D shows a schematic top view of the digital model 270 of the dental appliance 100, according to an embodiment of the present disclosure. At step 308, the method 300 includes incorporating the arrangement 120 of the plurality of features 130 in the one or more regions 262 of the initial digital model 260 to generate the digital model 270 of the dental appliance 100. In some embodiments, the digital model 270 of the dental appliance 100 may be represented in a CAD file or a 3D printable file, such as a STL file.

In some embodiments, incorporating the arrangement 120 of the plurality of features 130 in the one or more regions 262 of the initial digital model 260 includes merging the arrangement 120 of the plurality of features 130 in the one or more regions 262 of the initial digital model 260. In some embodiments, incorporating the arrangement 120 of the plurality of features 130 in the one or more regions 262 of the initial digital model 260 may include superimposing the arrangement 120 of the plurality of features 130 on the one or more regions 262 of the initial digital model 260. In some embodiments, incorporating the arrangement 120 of the plurality of features 130 in the one or more regions 262 of the initial digital model 260 may include aligning or orienting the arrangement 120 of the plurality of features 130 with the one or more regions 262 of the initial digital model 260. In some embodiments, the digital model 270 of the dental appliance 100 may be transmitted to a third party (e.g., a clinician office, a laboratory, a manufacturing facility, or other entity) for forming the dental appliance 100.

FIG. 9E shows an exemplary additive manufacturing apparatus 280 forming the dental appliance 100 based on the digital model 270 shown in FIG. 9D, according to an embodiment of present disclosure. At step 310, the method 300 includes forming the dental appliance 100 based on the digital model 270 of the dental appliance 100.

In some embodiments, forming the dental appliance 100 further includes additively forming the dental appliance 100. In some embodiments, the dental appliance 100 may be additively formed using an additive manufacturing technique, such as stereolithography (SLA) in which successive layers of material are laid down by the additive manufacturing apparatus 280 under control of a computer (not shown). In some embodiments, the computer may include a display and one or more user input devices, such as a mouse or a keyboard. In some embodiments, the additive manufacturing apparatus 280 may also include an input device or an output device, such as a control input (e.g., button, touchpad, thumbwheel, etc.), or a display (e.g., LCD or LED display) to provide status information. Some other examples of additive manufacturing techniques include Fused Filament Fabrication (FFF), Powder Bed Fusion (PBF), and the like. Therefore, the materials used for forming the body 110 of the dental appliance 100 may be varied conveniently to suit applications having varying color, shape, and stiffness requirements.

Further, additively forming the dental appliance 100 may reduce or eliminate the need of support structures are otherwise required to form the dental appliance 100 using conventional manufacturing techniques. This may reduce the complexity as well as a time required to form the dental appliance 100. Further, it may also reduce a material requirement to form the dental appliance 100. This may reduce a cost of forming the dental appliance 100. Additionally, this may reduce waste generation as the support structures are typically removed after the dental appliance 100 is formed. Therefore, additively forming the dental appliance 100 may reduce complexity of manufacturing the dental appliance 100 and also be environmentally sustainable.

Moreover, additively formed dental appliance 100 may be precise and have a smooth finish. Further, designing the dental appliance 100 by modifying the digital model 270 may be simpler for the dental practitioner. Further, forming the dental appliance 100 based on the digital model 270 may be convenient and faster than the other manufacturing techniques.

In some other embodiments, forming the dental appliance 100 further includes subtractively forming the dental appliance 100. In some embodiments, the dental appliance 100 may be formed by milling. In some embodiments, the dental appliance 100 may be formed from a blank (not shown). The blank generally refers to a solid block of material from which the dental appliance 100 can be machined. In general, the blanks are attached to a support, a stub, or a mandrel that fits into a milling machine (not shown). In some embodiments, the blank may have a rough shape of the contour 113 of the dental appliance 100.

In some embodiments, forming the dental appliance 100 may include additively and subtractively forming the dental appliance 100. For example, an initial dental appliance (not shown) may be formed additively based on the initial digital model 260 and the arrangement 120 of the plurality of features 130 may be formed subtractively to form the dental appliance 100. For example, after the initial dental appliance has been formed, the dental appliance 100 may be formed by forming the arrangement 120 of the plurality of features 130 by CNC or robotic machinery, such as, end mill or LASER cutter.

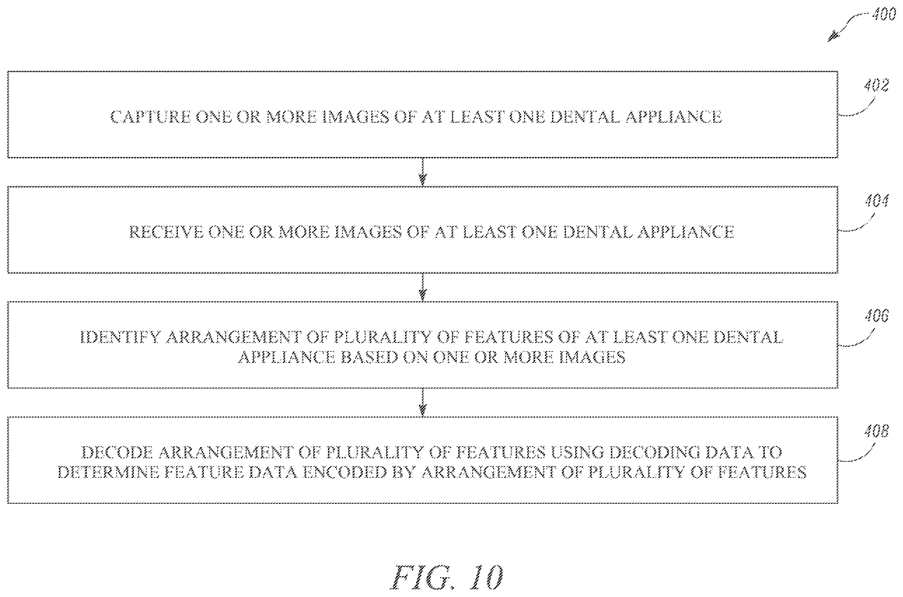

FIG. 10 illustrates a flowchart illustrating a method 400 of identifying the dental appliance 100 for the patient 10, according to an embodiment of the present disclosure.

The method 400 will be described with reference to FIGS. 1 to 8. The method 400 includes the following steps:
At step 402, the method 400 includes capturing the one or more images 212 of the at least one dental appliance 100.
At step 404, the method 400 includes receiving the one or more images 212 of the at least one dental appliance 100.
At step 406, the method 400 includes identifying the arrangement 120 of the plurality of features 130 of the at least one dental appliance 100 based on the one or more images 212.
At step 408, the method 400 includes decoding the arrangement 120 of the plurality of features 130 using the decoding data to determine the feature data 140 encoded by the arrangement 120 of the plurality of features 130.

As discussed above, in some embodiments, the at least one dental appliance 100 includes the plurality of dental appliances 100 corresponding to the plurality of corresponding patients 10. In some embodiments, upon decoding the arrangement 120 of the plurality of features 130 of each of the plurality of dental appliances 100, the method 400 further includes grouping the plurality of dental appliances 100 according to the feature data 140.

Unless otherwise indicated, all numbers expressing feature sizes, amounts, and physical properties used in the specification and claims are to be understood as being modified by the term “about”. Accordingly, unless indicated to the contrary, the numerical parameters set forth in the foregoing specification and attached claims are approximations that can vary depending upon the desired properties sought to be obtained by those skilled in the art utilizing the teachings disclosed herein.

Although specific embodiments have been illustrated and described herein, it will be appreciated by those of ordinary skill in the art that a variety of alternate and/or equivalent implementations can be substituted for the specific embodiments shown and described without departing from the scope of the present disclosure. This application is intended to cover any adaptations or variations of the specific embodiments discussed herein. Therefore, it is intended that this disclosure be limited only by the claims and the equivalents thereof.

1.-8. (canceled)

9. A system comprising:
at least one dental appliance for a corresponding patient, the at least one dental appliance comprising:
a body comprising a first surface and a second surface opposite to the first surface, the second surface defining a channel for receiving a plurality of teeth of the corresponding patient; and
an arrangement of a plurality of features disposed on the first surface of the body, each of the plurality of features comprising:
a protrusion extending from the first surface opposite to the second surface;
a depression extending partially from the first surface towards the second surface; or
a through-hole extending from the first surface to the second surface;
wherein the arrangement of the plurality of features encodes a feature data representative of a dental appliance information of the at least one dental appliance or a patient information of the corresponding patient;
an imaging device;
a processor communicably coupled to the imaging device;
a memory communicably coupled to the processor, the memory storing an algorithm for decoding the arrangement of the plurality of features of the at least one dental appliance, wherein the processor is configured to:
control the imaging device to capture one or more images of the at least one dental appliance;
receive the one or more images of the at least one dental appliance from the imaging device;
identify the arrangement of the plurality of features of the at least one dental appliance based on the one or more images; and
decode the arrangement of the plurality of features using the decoding data to determine the feature data encoded by the arrangement of the plurality of features; and
wherein the processor is also configured to execute a machine learning model that is trained to receive the one or more images of the at least one dental appliance from the imaging device and identify the arrangement of the plurality of features of the at least one dental appliance based on the one or more images.

10. (canceled)

11. The system of claim 910, wherein the at least one dental appliance comprises a plurality of dental appliances corresponding to a plurality of corresponding patients, and wherein, upon decoding the arrangement of the plurality of features of each of the plurality of dental appliances, the processor is further configured to group the plurality of dental appliances according to the feature data.

12. (canceled)

13. The system of claim 912, wherein the machine learning model is further trained to decode the arrangement of the plurality of features using the decoding data to determine the feature data.

14. The system of claim 9, wherein the first surface comprises an occlusal surface of the at least one dental appliance or an incisal surface of the at least one dental appliance.

15. The system of claim 9, wherein at least one feature of the plurality of features has a different shape or different dimensions than other features of the plurality of features.

16. The system of claim 910, wherein at least one feature of the plurality of features has a circular shape, an elliptical shape, a triangular shape, a rectangular shape, or a polygonal shape.

17. The system of claim 16, wherein the imaging device is configured to capture the one or more images in a single projection plane.

18. The system of claim 9, wherein the first surface comprises a plurality of teeth regions corresponding to and aligned with the plurality of teeth of the corresponding patient, and wherein the plurality of features is arranged in at least two teeth regions of the plurality of teeth regions.

19. The system of claim 9, wherein the dental appliance information comprises at least one of a production batch information of the at least one dental appliance, a material information of the at least one dental appliance, and a manufacturing timestamp information of the at least one dental appliance.

20. The system of claim 940, wherein the patient information comprises at least one of an identification information of the corresponding patient, an address information of the corresponding patient, a contact information of the corresponding patient, and a treatment stage information of the corresponding patient.

21. The system of claim 20, wherein the memory further stores at least one digital model corresponding to the at least one dental appliance, and wherein the processor is configured to:
decode the arrangement of the plurality of features using the decoding data to determine the treatment stage information of the corresponding patient;
determine a contour of at least one portion of the first surface of the at least one dental appliance based on the one or more images;
compare the contour of the at least one portion of the first surface with the at least one digital model; and
determine the identification information of the corresponding patient based on the comparison.

22.-27. (canceled)
